(19) United States
(12) Patent Application Publication
(10) Pub. No.: US 2014/0358215 A1
(43) Pub. Date: Dec. 4, 2014

(54) APPARATUS FOR DEPLOYING A STENT GRAFT

(71) Applicants: Mark Baylis, Didcot Oxfordshire (GB); Duncan Keeble, Didcot Oxfordshire (GB)

(72) Inventors: Mark Baylis, Didcot Oxfordshire (GB); Duncan Keeble, Didcot Oxfordshire (GB)

(73) Assignee: Lombard Medical Limited, Didcot Oxfordshire (GB)

(21) Appl. No.: 14/009,583

(22) PCT Filed: Mar. 30, 2012
(86) PCT No.: PCT/GB2012/050724

§ 371 Date: Apr. 3, 2014

(30) Foreign Application Priority Data
Apr. 8, 2011 (GB) 1106017.5

(51) Int. Cl. A61F 2/962

(52) U.S. Cl. CPC A61F 2/962
USPC 623/1.12

(57) ABSTRACT

Apparatus for deploying a tubular medical (200) device in vivo comprises an elongate element (10) for passing into the bore of the medical device, a deployment device having at least one arm for engaging with the medical device, the arm being moveable in a radial direction relative to the longitudinal axis of the elongate element from a first position to a second position, the second position being spaced radially further from the elongate element than the first position, and a thread (60) for the arm, said thread being associated with the arm such that pulling on the thread moves said arm from the second position towards the first position, whereby in use movement of the arm from the first position to the second position enables radial deployment of the medical device.

The present invention relates to apparatus and methods for deploying a tubular medical device, and in particular an implantable stent graft.

An endovascular stent-graft is designed is exclude the flow of blood to an aneurysm that has been formed within the wall of the lumen (for example the aorta). This is achieved by accessing the aneurysm via an artery, usually within the patient's leg, with a system designed to deliver, position and deploy the stent graft so that it bridges and seals off the aneurysm.

A stent graft is a (usually) tubular device with walls made from a flexible sheet material, supported by a rigidising frame (the stent) which may be formed from a super-elastic metal such as a shape memory alloy (commonly nitinol). In some designs, such as the Ovation (Trivascular Inc), the rigidising frame is added after the flexible tubular sheet component has been put in position by, for example, filling channels formed in the tubular sheet with a fluid which becomes rigid. Some stent graft designs are fixed to the aorta wall by means of barbs or hooks. The rigidising frame maintains the tubular shape of the stent graft, while providing a radial sealing force to create a proximal and distal seal with the aortic wall.

In order to deliver a stent graft to the locus of the aneurysm, it is conventionally collapsed (that is, reduced in diameter), loaded on a delivery catheter and delivered to the aneurysm where it is positioned and deployed by expanding its diameter, or otherwise dilating, to seal off the aneurysm as described above.

The stent frame can be manufactured from a multiply perforated tube of rigid material with a first narrow diameter. Upon dilation of the frame by external means, such as an endoluminal balloon, the perforations allow significant plastic deformation of the material to take place, causing the stent to adopt and maintain a second, wider, diameter. In a second method of making a stent frame, the frame may be formed of a plurality of resilient struts made from, for example, stainless steel or from Elgiloy, which are connected at their ends to provide a self-expandable frame (for example the Gianturco “Z-stent” marketed by Cook, Inc). Alternatively, the stent frame may use a shape-memory alloy, such as Nitinol, to provide a resilient or thermally initiated expansion of the stent. Examples of Nitinol stents are found in the Anaconda™ device marketed by Terumo and the Aorfix™ device marketed by the present applicant and disclosed in WO 99/37242, the contents of which are incorporated herein by reference. Self-expanding stents have one stable shape when unconstrained which is their maximum diameter. They are deformed under compression and then expand automatically when the compression is removed.

Although self-expanding stents expand within the aorta automatically when compression is removed (for example when they are ejected from a delivery sheath), it is necessary to control the deployment carefully to ensure that the stent graft is accurately positioned relative to the aneurysm. For example, the Anaconda™ device is deployed by means of a complicated system of threads and wires which are used to manipulate the mouth of the stent graft from the side stent graft proximate the heart and which then must be removed from the stent graft after deployment in order that the deployment system can be removed from the patient's body.

U.S. Pat. No. 5,713,907 (Endotex Interventional Systems, Inc.) discloses a device having an expandable frame for deploying an expandable stent graft. The frame can be advanced and retracted (and thereby expanded and contracted) by the surgeon pushing or pulling on a deployment shaft. The stent graft may be deployed incrementally. However, the disclosed device does not allow a stent graft easily to be repositioned in the body lumen.

US 2008/0300667 (Hebert) discloses a delivery system for an expandable stent which employs a flexible arm which is resiliently biased outwardly in order to pin the proximal end of the stent against the internal surface of the delivery catheter and prevent it from deploying while the distal end of the stent is being deployed.

There is a need for a deployment system which is capable of accurate deployment and positioning of a stent graft but which can easily be detached from the stent graft and removed from the patient's body without the risk of snagging.

In accordance with a first aspect of the invention, there is provided apparatus for deploying a tubular medical device in vivo, comprising an elongate element for passing into the bore of the medical device, a deployment device having at least one arm for engaging with the medical device, the arm being moveable in a radial direction relative to the longitudinal axis of the elongate element from a first position to a second position, the second position being spaced radially further from the elongate element than the first position, and a flexible element for the arm, said flexible element being associated with said arm such that pulling on the flexible element moves said arm from the second position towards the first position, whereby in use movement of the arm from the first position to the second position enables radial deployment of the medical device.

In accordance with a second aspect of the invention, there is provided apparatus for deploying a tubular medical device in vivo, comprising an elongate element for passing into the bore of the medical device, and a deployment device having at least one arm for engaging with the medical device, the arm being moveable in a radial direction relative to the longitudinal axis of the elongate element from a first position to a second position, the second position being spaced radially further from the elongate element than the first position, whereby in use movement of the arm from the first position to the second position enables radial deployment of the medical device, and wherein the arm has means for removably attaching the arm to the wall of the medical device.

In accordance with a third aspect of the invention, there is provided apparatus for deploying a tubular medical device in vivo, comprising an elongate element for passing into the bore of the medical device, and a deployment device having two arms for engaging with the medical device, the arms being moveable independently in a radial direction relative to the longitudinal axis of the elongate element from a first position to a second position, the second position being spaced radially further from the elongate element than the first position, whereby in use movement of at least one of the arms from the first position to the second position enables radial deployment of the medical device.

The provision of an elongate element and a deployment arm enables the apparatus to be centred in the body lumen (for example by mounting the elongate element on a guide wire) whilst deploying a tubular medical implant.

In a preferred embodiment, the deployment device has at least two arms which may be operated independently to enable a stent graft (for example) to be deployed within an artery. Alternatively, the elongate element may take the function of one of the arms.

The arm or arms are preferably resiliently biased into the second (open) position which enables the apparatus to be used to deploy stents or stent grafts which are not self-expanding (that is, which require some assistance to be expanded) for instance where a tubular sheet component is to be placed before placing or creating the rigidising element. However, in an alternative embodiment the arm(s) could be unbiased either way or even biased radially inwardly (although this is not preferred).

In a preferred embodiment, the apparatus comprises a flexible element (such as a thread) for each arm, said flexible element being associated with said arm (for example looped around said arm) such that pulling on the flexible element moves said arm from the second position to the first position.

The apparatus may also comprise means for redirecting each flexible element from a radial direction relative to the elongate element to a longitudinal direction relative to the elongate element. The means for redirecting is preferably generally toroidal in shape and is mounted on the elongate element; it may have at least one channel therein for accepting at least one flexible element.

Accordingly, in a preferred embodiment, each flexible element passes from the associated arm, around the means for redirecting and towards the end of the elongate element distal to the deployment device. However, in an alternative embodiment each flexible element passes from its arm, around the means for redirecting and towards the end of the elongate element proximate the deployment device, before turning 180 degrees and then being directed towards the end of the elongate element distal to the deployment device. In either embodiment the flexible element(s) may pass at some point through the wall of centre catheter 10 and into bore 15.

In a fourth aspect of the invention, there is provided a method of deploying a tubular medical device comprising the steps of:
(i) providing a tubular medical device mounted on apparatus as defined above, wherein the elongate element is at least partially inside the bore of the medical device and the at least one arm is engaged with the medical device, and wherein the medical device is inserted into a delivery sheath so as to be constrained in a collapsed configuration,
(ii) positioning the medical device to a locus of an aneurysm,
(iii) at least partly removing said delivery sheath in order to allow at least a part of the device to move into an open configuration, whereby movement of said at least one arm from the first to the second position is controlled in order to control deployment of the medical device.
(iv) if required, tensioning at least one of the flexible elements to cause at least one arm to return from the second position to the first position in order to at least partially reverse the deployment of the medical device.
(v) re-positioning the medical device close to an alternative location
(vi) repeating steps iii, iv and v until a satisfactory position has been achieved
(vii) completing deployment of the medical device, including disconnection of the device from the at least one arm.

Thus, the apparatus of the present invention can be employed to test, control and perfect accurate placement and deployment of medical devices in the lumen of a vessel, more particularly endovascular stent grafts. The apparatus is of particular value in situations requiring particularly exact placement and control of the implant, examples of which include:
(i) When placing a stent graft in an artery where there is a very short region in which the stent graft can gain adequate fixation and seal, for example where this distance between the renal arteries and the top (cephalad) margin of the aneurysm sac is less than 20 mm long.
(ii) When placing a stent graft in an artery where the region in which the stent graft can gain adequate fixation and seal is sharply angled.
(iii) Where particular features on the surface of the stent graft, such as troughs, cut-aways, fenestrations or side branches, require precise control to align these features with corresponding features, such as branch vessels, in the patient's anatomy.
(iv) When placing a stent graft in stages in which a flexible tube component is placed before a rigidising element is introduced, the arms can be used to open the said tube. Without such opening means, blood flow would force the flexible tube to collapse or require a self expanding component to be pre-attached to the said tube.

A number of preferred embodiments of the present invention will now be illustrated by way of example, with reference to the drawings, in which:

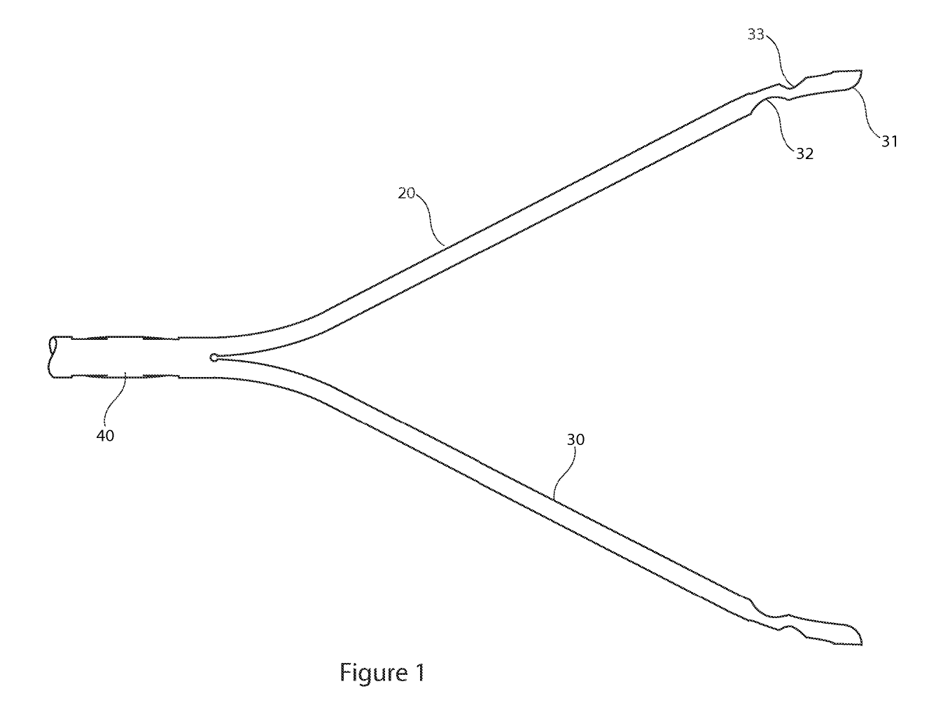

FIG. 1 is a plan view of a Y-piece deployment device in accordance with the invention;

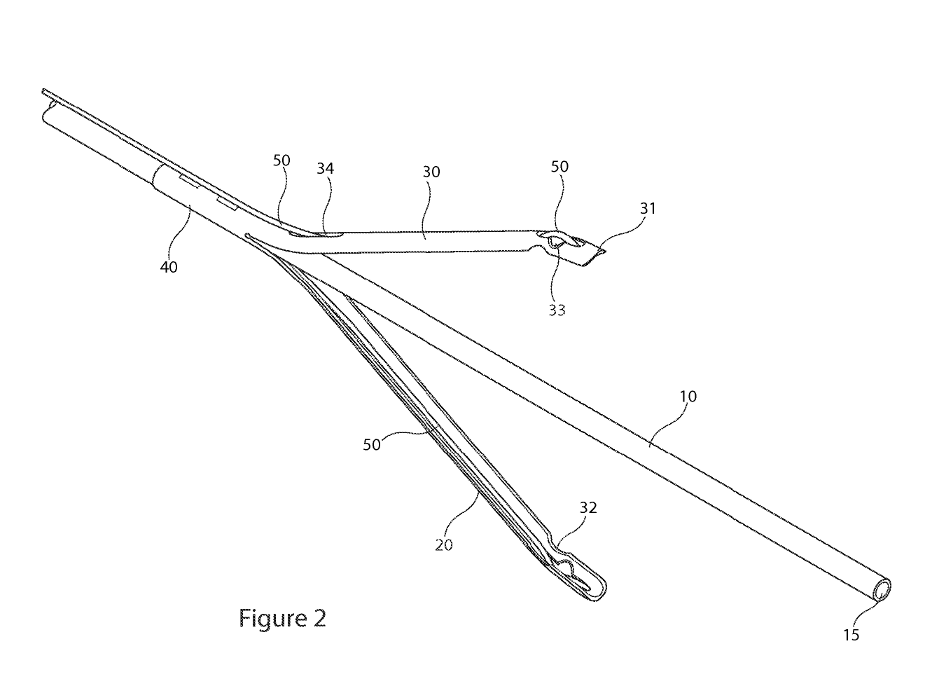

FIG. 2 is a perspective view of apparatus in accordance with the invention for deploying a medical device;

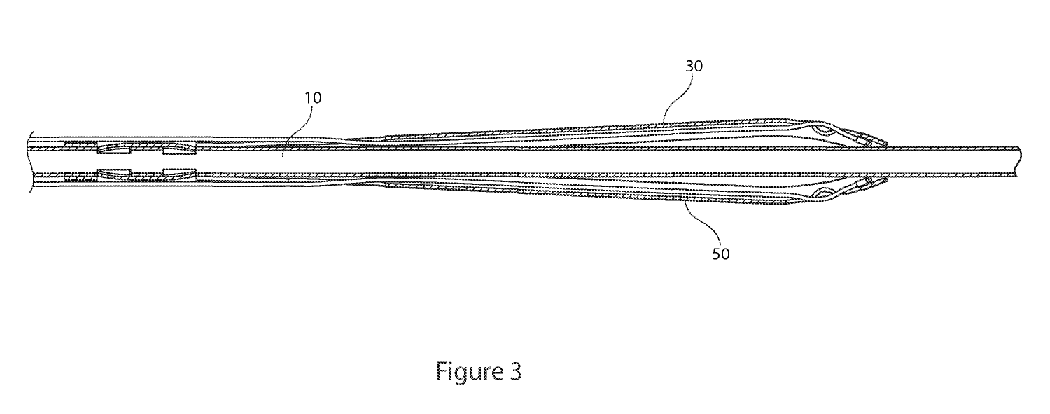

FIG. 3 is a side view of the apparatus for FIG. 2 shown in a collapsed configuration;

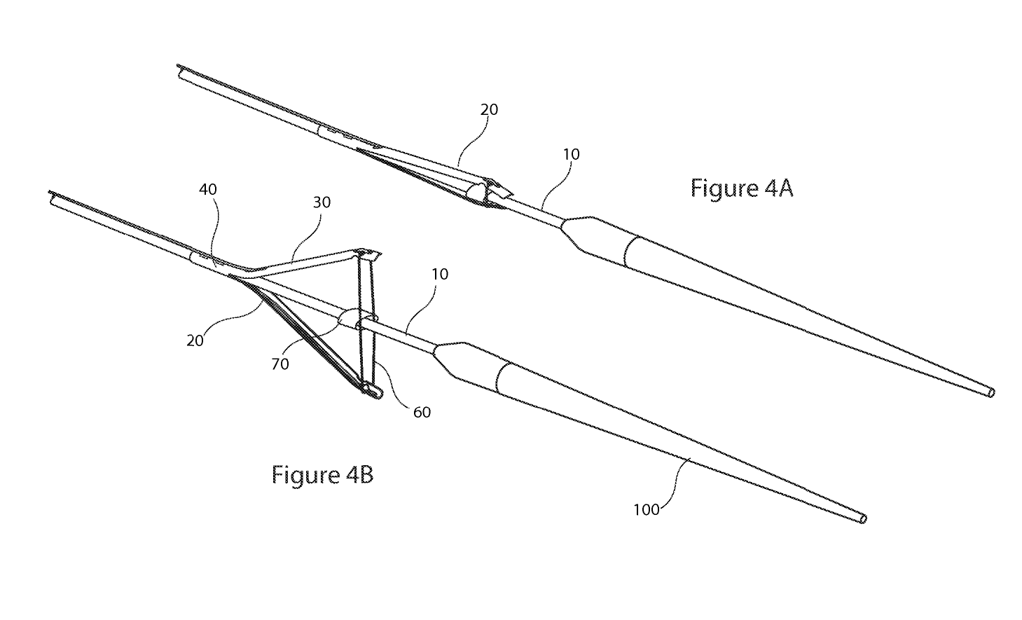

FIGS. 4A and 4B are further perspective views of the apparatus for FIG. 2 shown in collapsed and open configuration respectively;

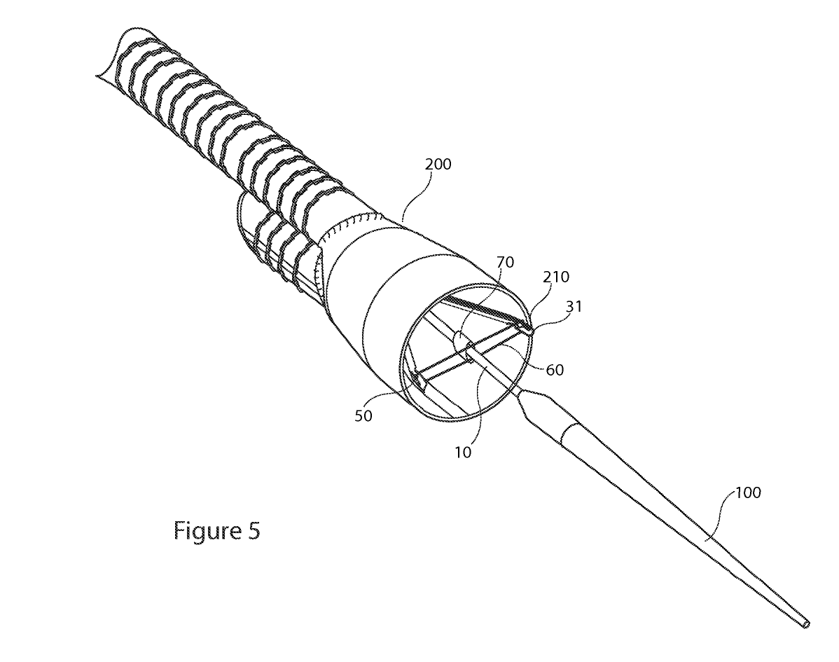

FIG. 5 is a perspective view of a stent graft mounted on the apparatus for FIG. 2 in an open configuration; and

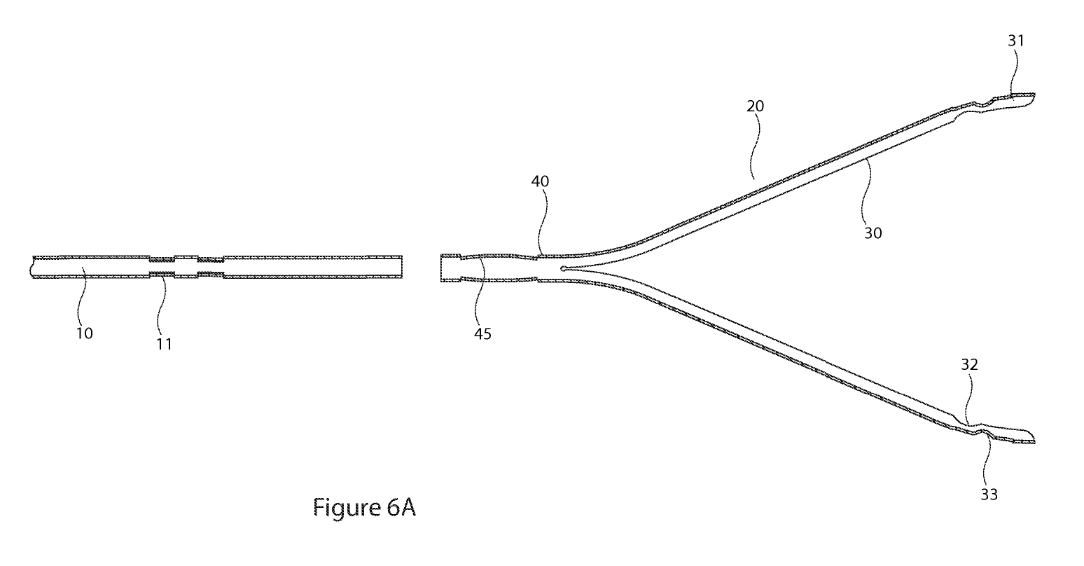

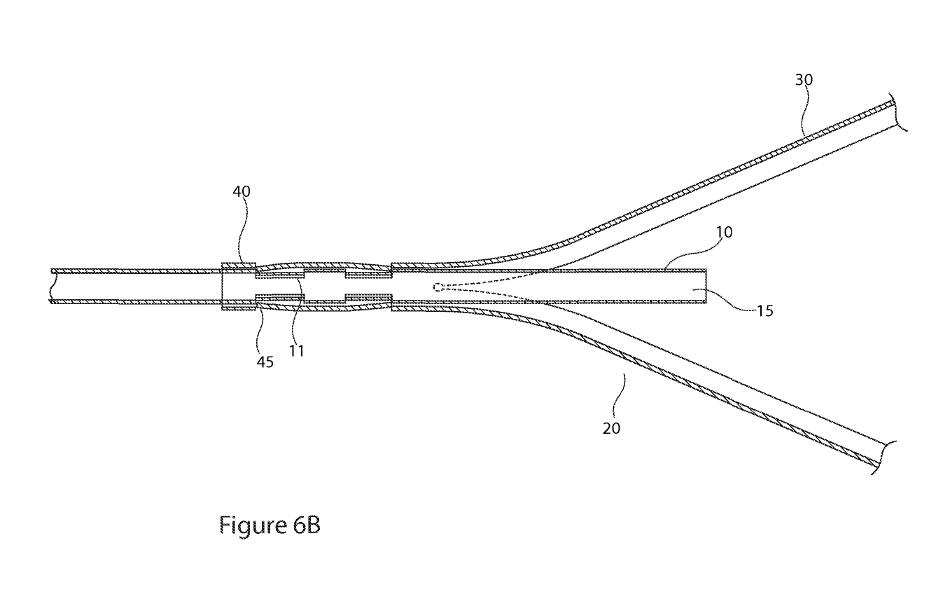

FIGS. 6A and 6B are views in cross-section showing the way in which the Y-piece fits onto the centre catheter.

Turning to FIG. 1, Y-piece 20 is formed of nitinol and comprises two arms 30 joined to cylindrical stem 40, arms 30 being biased into an open configuration. Three pieces of nitinol can be joined together to form each arm 30, which terminates in end piece 31 having cutaway section 32, aperture 33 and slot 34, the functions of which will be explained below. Alternatively, the Y-piece can be of mono-bloc construction.

Centre catheter 10 (see FIG. 2) has delivery bore 15 through which the apparatus can be threaded on a guide wire (not shown). Cylindrical stem 40 of Y-piece 20 is mounted on centre catheter 10 as will be described in more detail with reference to FIGS. 6A and 6B. Each arm 30 has an associated release wire 50 which passes along the outside of the centre catheter 10, through slot 34 and along the inside of arm 30. Release wire 50 then passes through aperture 33 to form a loop on the outside face of each arm 30 before passing back through the aperture 33 to lie flush with the inner surface of arm 30 at end section 31.

As mentioned above, Y-piece 20 is formed from nitinol and this is preferably shape set such that arms 30 are biased into the open configuration as shown in FIGS. 1 and 2. The elastic properties of nitinol are such that arm 30 can be resiliently compressed in a radial direction towards centre tube 10 so as to lie substantially flush against centre tube 10 as shown in FIG. 3. If the arms are formed from a single tube then the inside face of each arm will be curved so as to pack tightly to the sides of the centre catheter. It will be appreciated that the slight bow in each arm 30 means that they are not completely flat with respect to centre catheter 10 but can be compressibly flattened. Nevertheless this is sufficient to enable the apparatus to be passed down the lumen of an artery so as to deliver a mounted stent graft to the locus of an aneurysm as will be described below.

The mechanism for moving Y-piece 20 between an open and collapsed configuration is shown in the perspective views of FIG. 4A and 4B. In particular, each arm 30 has associated with it a deployment thread 60 which may be looped over the end section 31 and/or through aperture 33. Deployment thread 60 then passes through a channel (not shown) in an annular olive 70 which is mounted on centre catheter 10, whereafter thread 20 passes back along (or optionally in) centre catheter 10 to the end of centre catheter 10 proximate the user of the apparatus.

It will be appreciated that when the user pulls on the ends of deployment thread 60 this serves to pull the associated arm 30 against its bias and to move it into the collapsed configuration. It will further be appreciated that each of the two arms 30 may be operated independently, which can be useful when deploying, for example, a stent graft within the thoracic arch.

FIG. 5 shows stent graft 200 mounted on apparatus in accordance with the invention in the open configuration. It can be seen that stent graft 200 is mounted on centre catheter 10 and aligned with Y-piece 20 such that end sections 31 of arms 30 line up with mouth 210 of stent graft 200. Release wires 50 are each pierced through the wall of stent graft 200 and mouth 210 and then rethreaded through aperture 33 so as to attach stent graft 200 to each of arms 30 at opposite sides of mouth 210.

Is use, stent graft 200 is mounted on the deployment apparatus as shown in FIG. 5. It is then collapsed and loaded into a delivery sheath (not shown) by any suitable method and delivered to the locus of an aneurysm. Once in place, it is deployed by retracting the delivery sheath and controlling the expansion of mouth 210 by maintaining (and slowly releasing) inwards tension on delivery arms 30 by pulling on deployment threads 60.

When stent graft 200 is in place (which may involve barbs—not shown—being implanted into the artery wall) then the deployment apparatus can be detached from stent graft 200 by pulling on release wires 50 to unthread them from the walls of stent graft 200 so as to detach arms 30. Arms 30 can then be moved into the collapsed configuration by pulling on threads 60 and Y-piece 20 and centre catheter 10 can be removed from the bore of stent graft 200.

Turning finally to FIG. 6A and 6B, these show in more detail the way in which Y-piece 20 fits onto centre catheter 10. It can be seen that stem 40 has a number of tabs 45 laser cut into the main shaft which are heat set to protrude into the bore of stem 40. Centre catheter 10 has a number of laser cut slots 11 which are designed to accept tabs 45. Thus in use stem 40 is positioned over centre catheter 10 by opening tabs 45 out and pushing stem 40 into place so that tabs 45 engage with the slots 11 to prevent axial or circumferential movement of Y-piece 20.

1-28. (canceled)

29. A placement device for deploying a tubular medical device in a body, the placement device including:
an elongate element for passing into a bore within the medical device,
a deployment device having an arm for engaging with the medical device, the arm being moveable in a radial direction relative to a longitudinal axis of the elongate element from a first position to a second position, the second position being spaced radially further from the elongate element than the first position, and
a flexible element for the arm, the flexible element being associated with the arm such that pulling on the flexible element moves the arm from the second position towards the first position,
whereby in use, movement of the arm from the first position to the second position enables radial deployment of the medical device.

30. The placement device of claim 29 wherein the arm is resiliently biased into the second position.

31. The placement device of claim 29 wherein the deployment device has at least two of the arms, each arm being independently moveable in a radial direction relative to the longitudinal axis of the elongate element.

32. The placement device of claim 31 wherein the arms are circumferentially spaced at least substantially evenly around the elongate element.

33. The placement device of claim 31 wherein the arms are substantially identical in construction.

34. The placement device of claim 29 wherein the flexible element includes at least one thread.

35. The placement device of claim 34 wherein the flexible element is a single thread which is looped around the arm.

36. The placement device of claim 29 wherein the elongate element is tubular, and wherein the flexible element extends through the elongate element between the arm and an end of the elongate element distal to the deployment device.

37. The placement device of claim 29 additionally including means for redirecting the flexible element from a radial direction relative to the elongate element to a longitudinal direction relative to the elongate element.

38. The placement device of claim 37 wherein the means for redirecting is generally toroidal in shape and is mounted on the elongate element.

39. The placement device of claim 37 wherein the means for redirecting has at least one channel therein for accepting at least one flexible element.

40. The placement device of claim 37 wherein the flexible element extends from the arm, around the means for redirecting, and towards an end of the elongate element distal to the deployment device.

41. The placement device of claim 37 wherein the flexible element passes from the arm, around the means for redirecting, and towards an end of the elongate element proximate the deployment device, then turning 460 degrees to extend towards the end of the elongate element distal to the deployment device.

42. The placement device of claim 29 wherein the deployment device has at least two arms, each arm having a first end wherein the first ends of the arms are joined, and wherein each arm has an opposing second end engaged with the medical device.

43. The placement device of claim 42 wherein the deployment device includes a stem from which the arms project, and which in use is mounted on the elongate element.

44. The placement device of claim 43 wherein the stem is in the form of a hollow cylinder through which the elongate element extends.

45. The placement device of claim 43 wherein the stem has a tab formed from a shape memory alloy, the tab engaging the elongate element.

46. The placement device of claim 29 wherein the arm includes means for removably attaching the arm to a wall of the medical device.

47. The placement device of claim 46 wherein the means for removably attaching the arm includes a wire for piercing the wall.

48. The placement device of claim 47 wherein the arm has an aperture therein, and wherein in use the wire passes from one side of the arm through the aperture, through the wall, and then back through the aperture in order to attach the arm to the wall.

49. The placement device of claim 29 further including a tubular medical device mounted thereon, wherein the elongate element is at least partially inside a bore of the medical device and the arm is engaged with the medical device.

50. The placement device of claim 49 wherein the arm is in the second position and the medical device is in a radially open configuration.

51. The placement device of claim 49 wherein the arm is in the first position and the medical device is in a radially collapsed configuration.

52. The placement device of claim 51 further including a delivery sheath into which the tubular medical device is inserted so as to be constrained in a collapsed configuration.

53. A method of using the placement device of claim 52 to deploy the tubular medical device, the method including the steps of:
a. providing the placement device of claim 51,
b. delivering the tubular medical device to a locus of an aneurysm,
c. at least partly removing the delivery sheath in order to allow at least a part of the tubular medical device to move into an open configuration,
whereby movement of the arm from the first to the second position is controlled in order to control deployment of the tubular medical device.

54. The method of claim 53 wherein the movement of the arm is controlled by tension on the flexible element.

55. A deployment device having a stem with arms extending therefrom, the arms being moveable in a radial direction relative to a longitudinal axis of the stem from a first position to a second position, the second position being spaced radially further from the longitudinal axis of the stem than the first position.

56. A placement device for deploying a tubular medical device in a body, the placement device including:
an elongate element having a passage defined along its length;
a deployment device having the elongate element extending therefrom, and having two or more arms extending adjacent the elongate element, each arm being moveable in a radial direction relative to a longitudinal axis of the elongate element from a first position closely spaced to the elongated element to a second position spaced radially further from the elongate element than the first position;
two or more flexible elements, each extending through the passage of the elongated element to an associated one of the arms, wherein tension on one of the flexible elements moves its associated arm between the first and second positions.
